SEQUENCE LISTING (1 sequence)

SEQ ID NO: 1 - DNA, 292 bp, Homo sapiens
caggccctgtcacttggccccgccctgtccgccggccccgcccctgattccccgtccctgcgcctcgctggccggcgccgtggaggggcgggtccgggggcggggcgagggacggggcgggacgggctctgggtcccgcgcggccgctgaggggctgggagccgcggcggggcggtgcgagggcgggccggggccggttccgcgcgcagggattttaaatgtcccgctctgagccgggcgcaggagcagccggcgcggccgccagcgcggtgtagggggcaggcgcggatcc

(19) United States
(12) Patent Application Publication
(10) Pub. No.: US 2008/0260725 A1
(43) Pub. Date: Oct. 23, 2008

(54) Tag and target delivery system

(76) Inventors: Ulhas Naik, Hockessin, DE (US); Peter G. Millili, Cherry Hill, NJ (US)

Correspondence Address: RATNERPRESTIA, P.O. BOX 1596, WILMINGTON, DE, 19899, US

(21) Appl. No.: 12/079,681

(22) Filed: Mar. 28, 2008

Related U.S. Application Data
(60) Provisional application No. 60/921,119, filed on Mar. 29, 2007.

(51) Int. Cl. A61K 9/14; A61K 31/7088; A61K 39/395; C12N 15/00; A61P 35/04

(52) U.S. Cl. 424/130.1; 424/489; 514/44; 435/455; 977/773

(57) ABSTRACT

A system for targeting dividing cells and delivering therapeutic agents to the targeted cells using a Tag nanoparticle. Each Tag nanoparticle comprises a nucleotide encoding a Tag, which is expressed only in dividing cells. The system optionally utilizes a microparticle to deliver Tag nanoparticles to cells. Therapeutic nanoparticles comprising therapeutic compounds are subsequently targeted to the tagged cells via surface molecules that specifically bind to the Tag. The system may be adapted for specific patient needs, for example, targeting and eliminating cancer cells, preventing metastasis and secondary tumors, stimulating or inhibiting proliferation of endothelial cells, and stimulating cell regeneration in diseased organs such as liver.

### RELATED APPLICATIONS

This application claims priority to U.S. Provisional Application No. 60/921,119, which was filed on Mar. 29, 2007.

### GOVERNMENT SUPPORT

This invention was made with Government support under DGE-0221651, awarded by the National Science Foundation. The Government has certain rights in this invention.

### BACKGROUND OF THE INVENTION

Although some progress has been made in developing targeted cancer therapy, surgery and systemic chemo- and radiation therapy remain the chief methods for the treatment of cancer. These methods are highly invasive, damage healthy tissues and cells, and are accompanied by severe side effects that hinder recovery of the patient. In addition, these methods cannot control metastasis, formation of secondary tumors, and cancer recurrence.

Targeting drugs to diseased cells spares healthy cells, requires less drug, and reduces drug-toxicity. Procedures for drug targeting have employed a variety of targeting agents to selectively recognize cancer cells, including antibodies, asialoglycoprotein receptors, transferrin, transcobalamin, growth factor receptors, low density lipoprotein, and coated magnetic nanoparticles. The targeting agent may be conjugated to the anti-cancer agent, or the anti-cancer agent may be encapsulated in a liposome or other particle which bears a targeting agent. Anticancer agents have included cytotoxins, apoptosis-inducing compounds, antisense nucleotides, nucleotides encoding anticancer or therapeutic agents, and heat- and photo-sensitizers. (U.S. Pat. Nos. 6,749,863; 7,001,991; 7,005,139; 7,074,175). Drug targeting procedures have also been utilized to treat cells infected with micro-organisms and diseased cells other than cancer cells. (U.S. Pat. Nos. 7,101,842; 7,074,175).

However, none of these methods addresses the problems of metastasis, secondary tumor formation, or disease recurrence. Therefore, a need remains for a less invasive, targeted therapy that is easily adapted for specific disorders and for individual patients, and, which not only selectively treats diseased cells, but can also inhibit disease recurrence and provide simplified, targeted treatment of recurring disease.

### SUMMARY OF THE INVENTION

The invention comprises a composition and methods for tagging a dividing cell and targeted delivery of a therapeutic compound to a subject in need thereof. The invention has many uses in treating disease, including destruction of cancer cells, inhibition of metastasis, stimulation or inhibition of angiogenesis, and stimulation of tissue and organ regeneration.

More specifically, the composition for selectively tagging a dividing cell for subsequent targeted delivery of a therapeutic compound comprises one or more Tag nanoparticles, each of the Tag nanoparticles comprising a nucleotide operably linked to a promoter that functions only in dividing cells, wherein the nucleotide encodes a Tag peptide. The composition may further comprise a microparticle that contains the Tag nanoparticles.

The method of tagging a dividing cell for subsequent targeted delivery of a therapeutic compound to a subject in need thereof, comprises administering the composition to the subject.

The method for targeted delivery of a therapeutic compound to a subject in need thereof, comprises the steps of (1) tagging a dividing cell in the subject; and (2) administering to the subject a therapeutic nanoparticle comprising an effective amount of a therapeutic compound, wherein the therapeutic nanoparticle has an external surface comprising specific antibodies to the Tag peptide.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic drawing depicting the four stages of an embodiment of the Tag and Target delivery system.

FIG. 2 is a schematic drawing of an embodiment of a Tag vector.

FIG. 3 presents graphs demonstrating synchronization of the cell cycle in cultures of CHO cells (A); the effects of synchronization on expression of a transfected cyclin E-luciferase vector in CHO cells (B); and the effects of synchronization on expression of a transfected cyclin E-GFP vector in CHO cells (blue—background autofluorescence, red—lovastatin treatment, green—addition of mevalonic acid lactone following lovastatin treatment) (C).

FIG. 4 shows the size and shape characteristics of the DNA-bearing nanoparticles (A and B), and the cellular localization of DNA bearing nanoparticles in CHO cells (C).

FIG. 5 presents fluorescent micrographs demonstrating internalization of fluorescently tagged, DNA-bearing nanoparticles in CHO cells. (A) Cells transfected with unlabelled PEI-DNA nanoparticles; (B) Cells transfected with PNA-F alone; (C) Cells transfected with PEI-DNA-PNA-F nanoparticles.

FIG. 6 is a graph showing the effect of RGD peptide on the transfection efficiency of PEI-DNA-PEG nanoparticles.

FIG. 7 is a flow cytometry graph demonstrating the expression of a cyclin E-GFP vector administered via tail injection (green) or incorporated into nanoparticles injected into tumor tissue (red). The blue peak is background autofluorescence.

FIG. 8 is a fluorescence micrograph showing expression of the Cyclin E-GFP vector within tumor cells following injection of nanoparticles containing the vector into a solid tumor.

FIG. 9 is a fluorescence micrograph showing expression of HA-tagged JAM delivered by PEI-DNA nanoparticles on the surface of transfected CHO cells.

### DETAILED DESCRIPTION OF THE INVENTION

To support their characteristic rapid proliferation and growth, tumor cells produce growth factors that induce the formation of blood vessels (angiogenesis) around the tumor site. The new blood vessels provide a source of nutrients and other necessities to the tumor cells. One active area of cancer research is to find methods to inhibit angiogenesis and “starve” the tumor cells. However, some of these angiogenic factors not only induce formation of new blood vessels, but also increase the permeability of blood vessels in the area of the tumor, allowing larger molecules and particles to pass through the blood vessels in the vicinity of a tumor. In addition, these macromolecules and particles will be retained in tumor tissue for long periods, but are rapidly cleared from normal tissues. This phenomenon is referred to as the “enhanced permeability and retention effect” or “EPR effect” (Maeda, et al., Advances in Enzyme Regulation 41: 189-207, 2001; Yuan et al., Cancer Research 55: 3752-3756, 1995).

The EPR effect enhances the success of targeted tumor cell treatment, because it allows macromolecules and particles to exit blood vessels selectively in the vicinity of a tumor. The large molecules cannot exit the less permeable blood vessels that pass through normal tissue; therefore, macromolecules and particles carrying anti-tumor compounds concentrate near the tumor. As a result, the anti-tumor compounds are less likely to damage normal tissues and can be administered in lower concentrations.

In one embodiment of the present invention, the Tag and Target delivery system takes advantage of this property to selectively tag cancer cells for targeted delivery of anti-tumor compounds.

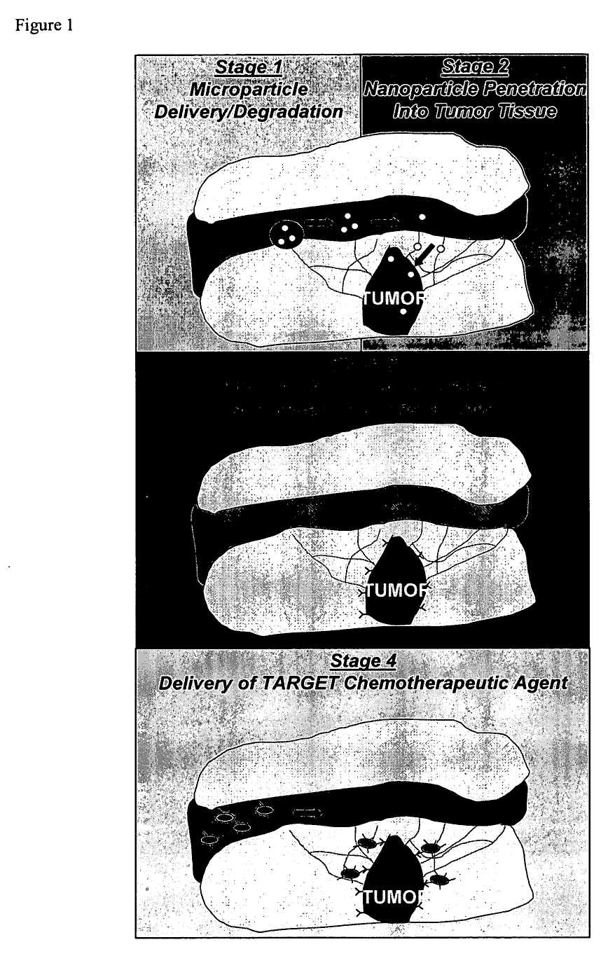

A generalized representation of one embodiment of the Tag and Target delivery system is shown in FIG. 1. In this embodiment, the system is administered to a subject in need of treatment for a cancerous epithelial cell tumor. First (Stage 1), a microparticle 1 comprising Tag nanoparticles 2 is introduced into the bloodstream of the subject. The microparticle attaches to the surface of a blood vessel 3 in the vicinity of the tumor 4 via surface antibodies that specifically bind to endothelial cell antigens. The microparticle then degrades and releases the Tag nanoparticles 2. The Tag nanoparticles bear surface antibodies to one or more tumor-cell antigens, and further comprise a nucleotide encoding a Tag peptide. In Stage 2, the Tag nanoparticles 2 exit the blood vessel in the vicinity of the tumor, bind to cells of the tumor 4 via the surface antibodies to tumor-cell antigens, and enter the tumor cells by endocytosis. In Stage 3, the endocytosed nucleotide encoding the Tag peptide enters the tumor cell nuclei and may or may not incorporate into the DNA of the subject. When the tumor cells divide, the Tag peptide 5 is expressed on the surface of each daughter cell. In Stage 4, a therapeutic nanoparticle 6 comprising a polymeric compound encapsulating a chemotherapeutic anti-cancer agent and bearing surface antibodies to the Tag peptide is administered to the subject. The therapeutic nanoparticle travels through the bloodstream, exits the blood vessel in the vicinity of the tumor, and binds specifically to the Tag peptide 5 on the tumor cells. The therapeutic nanoparticle then enters the tagged tumor cells via endocytosis, releases the anti-tumor agent inside the tumor cell, and destroys the tumor cell.

### Tag Nanoparticles

The nanoparticle comprises any biocompatible, endosomolytic, cationic construct, including, but not limited to, a liposome, hydrogel, or peptide-nucleic acid clamp. In one embodiment, the nanoparticle comprises a polymeric compound, including, but not limited to, a polyethylenimine, a poly(amidoamine), histidine, polyhistidine, polylysine, poly(D,L-lactide-co-glycolide), polyglycolic acid, and combinations thereof. A “biocompatible” polymer, as used herein, is a polymeric compound that is not toxic to cells. An “endosomolytic” polymer is a compound which is capable of escaping from an endosome inside a cell following endocytosis. The Tag nanoparticle further comprises a nucleotide operably linked to a promoter that is active only in dividing cells.

The Tag nanoparticle may be prepared by any known method for incorporating a nucleotide into a polymeric compound, as described, for example, in Boussif et al., P.N.A.S. USA 92: 7297-7301, 1995; Blessing et al., Bioconjugate Chem. 12: 529-537, 2001; Forrest, et al., Bioconjugate Chem. 14: 934-940, 2003; and Sullivan, et al., Gene Therapy 10: 1882-1890, 2003. One method is detailed below in Methods of Preparation 2. The Tag nanoparticle may have a diameter of 600 nanometers or less, and is preferably 50-400 nanometers in diameter.

The Tag nanoparticle may also bear external antibodies to surface antigens found on specific cell types. For example, the receptor for epidermal growth factor (EGFR) is over-expressed in many tumors. Antibodies to EGFR could be used to target the Tag nanoparticles to tumor cells and stimulate endocytosis of the Tag nanoparticles. Alternatively, EGF or EGF fragments could be incorporated into the nanoparticle to bind to EGFR on tumor cells and stimulate endocytosis, as described in Blessing et al., Bioconjugate Chem. 12: 529-537, 2001. Targeting antibodies and/or ligands are incorporated into the nanoparticle during its formation through any appropriate method, such as those described below for preparation of the microparticle.

In one embodiment of the invention the Tag nanoparticles are released from the microparticles in the vicinity of a tumor and exit the blood vessels through extravasation. In another embodiment, the Tag nanoparticles are directly administered intravenously. The nanoparticles specifically bind to target cells, e.g., tumor cells, via surface antibodies or ligands, and are taken into the target cells by endocytosis. The Tag nucleotide is subsequently released by degradation of the polymer and enters the nucleus of the target cell when the cell divides. The Tag nucleotide may integrate into the cell genome (stable transfection), or may not (transient transfection).

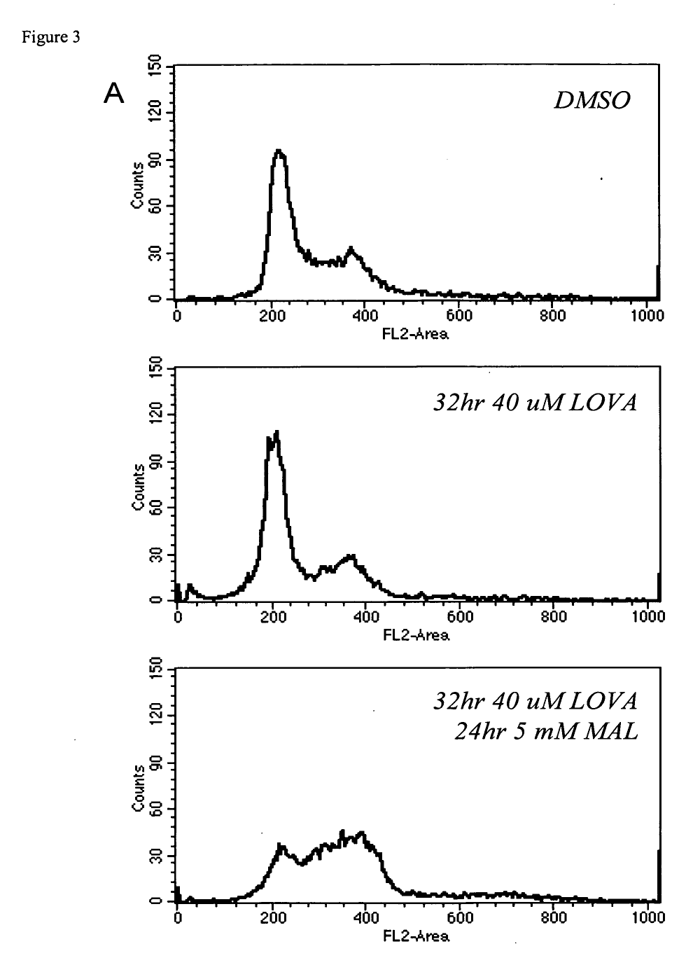

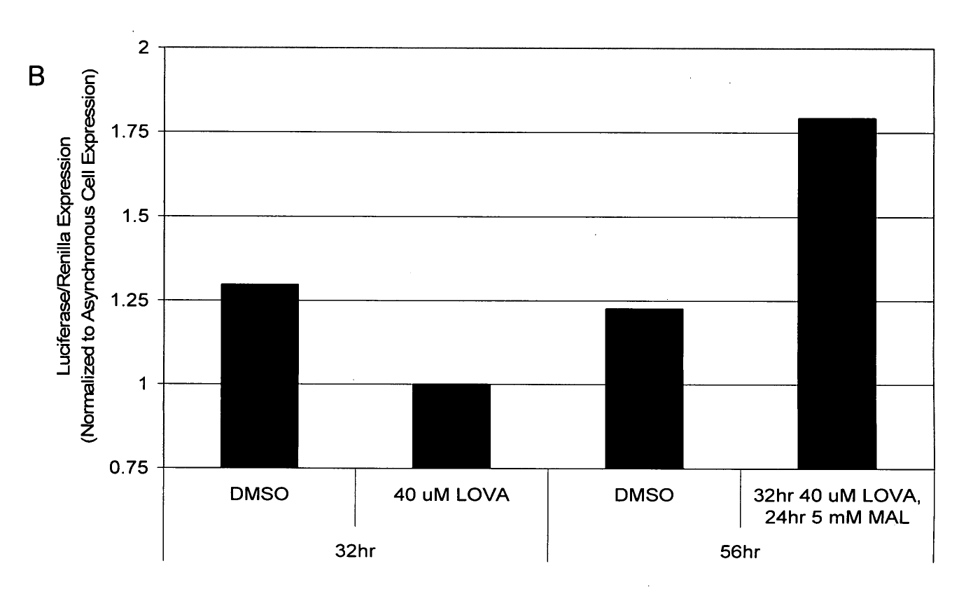

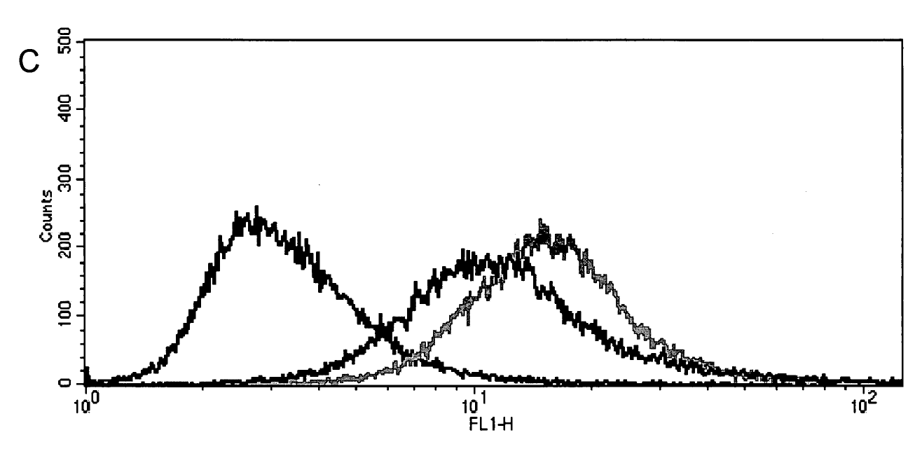

The nucleotide of the Tag nanoparticle is generally in the form of a nucleotide vector. Any appropriate vector capable of introducing the Tag nucleotide into the cells of interest may be utilized, including plasmid and viral vectors. The nucleotide vector includes a sequence encoding an appropriate Tag peptide. The coding sequence is operably linked to a promoter that is activated only after initiation of the mitotic cycle, such as upon entry into S phase. Suitable promoters include, but are not limited to, promoters for any cyclin and the promoter for E2F Transcription Factor. For example, in one embodiment, the nucleotide is based on the plasmid vector shown in FIG. 3, the pGL2 Basic luciferase reporter vector produced by Promega Corp., Madison, Wis. (Catalog no. E1641). The promoter for human cyclin E (GenBank Accession No. L48996; SEQ ID NO:1) is cloned into the multiple cloning site of the vector (Ohtani et al., P.N.A.S. USA 92: 12146-12150, 1995). DNA encoding the luciferase reporter gene (bp 76-1728) is excised and is replaced by the nucleotide sequence encoding the selected Tag peptide.

The selected promoter will be activated only in cells committed to divide. For example, the cyclin E promoter is activated only in the presence of high levels of the E2F1 transcription factor. (Ohtani et al., P.N.A.S. USA 92: 12146-12150, 1995). Therefore, the expression of sequences under the control of the cyclin E promoter will only be initiated in dividing cells. Because tumor cells are characterized by rapid proliferation, the promoter will be activated in the tumor cells and the Tag protein will be expressed as a surface protein on all daughter cells.

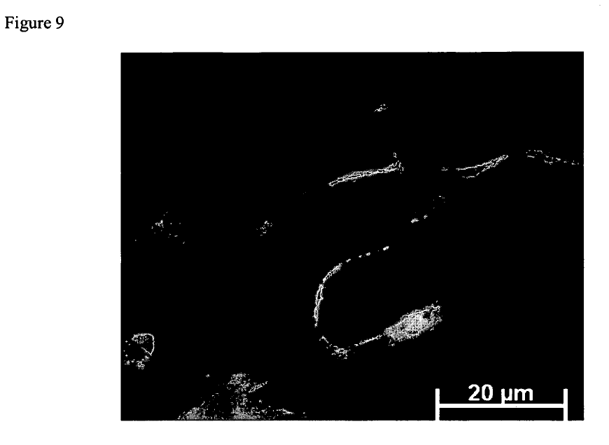

To ensure that the therapeutic compound is targeted specifically to the cells bearing the Tag, the Tag peptide is preferably a peptide that is not expressed by normal cells of the subject. The Tag peptide may be a xenogenic peptide, such as a bacterial or viral protein (including, but not limited to, n-methyl-D-aspartic acid receptor, muscarinic receptors, nicotinic receptors); a protein that cannot be accessed through the circulatory system, e.g., a peptide specific to cells of the brain, or a synthetic “nonsense” peptide such as the octapeptide, “FLAG®” (Sigma-Aldrich, St. Louis, Mo.). The Tag sequence is selected to ensure that the Tag peptide is expressed as a cell-surface molecule. In one embodiment, the Tag sequence encodes a fusion protein of junctional adhesion molecule A (JAM-A, aka JAM-1) (GenBank Accession No. AF172398) and FLAG® peptide. JAM-A is a cell-surface glycoprotein normally expressed on platelets (Naik, et al., J. Cell Science 114: 539-547, 2000). Fusion with the JAM-A sequence ensures that the selected Tag peptide will be expressed on the external surface of tumor cells, as shown in FIG. 9. In this experiment, the HA-JAM DNA construct described in Naik, et al. was transfected via PEI-DNA nanoparticles into CHO cells. Expression of hemagglutinin (HA) was demonstrated by immunodetection as described in Naik, et al. Other possible Tags may include, but are not limited to, viral phage coated proteins, yeast proteins, nuclear proteins, and Fas ligand.

The Tag peptide may also be a bacterial antigen selected to induce an immune response and aid in destruction of the tagged cell, such as those antigens described in Mayers et al., Comparative and Functional Genomics 4: 468-478, 2003. Such antigens include, but are not limited to lactadherin, tetanus toxin, carcinoembryonic antigen, bacterial surface proteins, and interferon-γ. Alternatively, the Tag peptide may be selected to induce an inflammatory response and thereby invoke heat stress. For example, interleukin-2 has been shown to increase the effectiveness of radiation and chemotherapy by stimulating “heat-stress” in the affected cells (Coffey, et al., JAMA 296: 445-448, 2006

### Microparticles

The microparticle serves as a delivery vehicle for nanoparticles containing the tagging system, and also reduces polymer cytotoxicity by controlling the release of the nanoparticles into a cell over time (De Rosa, et al., Journal of Pharmaceutical Sciences 91: 790-799, 2002). The microparticle may be synthesized from any biocompatible, biodegradable, polymeric compound according to any known methods, such as water-oil (W-O) and water-oil-water (W-O-W) emulsion methods, e.g., as described in Methods of Preparation 3, and in Freitas and Marchetti, Int. J. Pharmaceutics 295: 201-211, 2005; Jeyanthi, et al., J. Microencapsulation 14: 163-174, 1997; Sansdrap and Moes, J. Controlled Release 43: 47-58, 1997; Chung, et al., Int. J. Pharmaceutics 212: 161-169, 2001. As used herein, a “biodegradable” compound is one that may be broken down inside the body into components that can either be reused or disposed of with no significant associated toxicity. The nanoparticle comprising a Tag nanoparticle and a polymer is added to the organic (oil) phase, which is then mixed with the water phase and a surfactant. The mixture is emulsified by homogenization, and the organic solvent is then removed, e.g., by evaporation and/or extraction. The polymer microparticles are then washed in water and lyophilized under conditions that preserve their function, e.g., through the addition of glucose, as described in Werth, et al., Journal of Controlled Release 112: 257-270, 2006.

Biocompatible, biodegradable polymeric compounds include, but are not limited to, polyamides, polycarbonates, polyalkylenes, polyalkylene glycols, polyalkylene oxides, polyalkylene terepthalates, polyesters, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpyrrolidone.

In one embodiment, the microparticle may comprise poly(D,L-lactide-co-glycolide) (PLGA), and the Tag nucleotide may be complexed with polyethylenimine (PEI), for example, as described in Boussif et al., P.N.A.S. USA 92: 7297-7301, 1995. In this embodiment, an aqueous solution containing the Tag nucleotide complexed to PEI is emulsified with PLGA to form a W-O (water-oil) emulsion. This emulsion is added to an aqueous solution of poly(vinylalcohol) (PVA) and homogenized to form a W-O-W emulsion. Following evaporation of solvent, the microspheres are washed and freeze-dried. This procedure is described in detail in De Rosa, et al., Journal of Pharmaceutical Sciences 91: 790-799, 2002, which is incorporated herein by reference.

The microparticle may range in size from about 600 nanometers to about 10 micrometers in diameter, preferably about 1 to about 5 micrometers in diameter. This size range allows the microparticles to travel through capillaries, but prevents the microparticles from extravasating and entering the tumor tissue. The size of the microparticle is regulated by the concentration of polymer in the organic phase, the volume ratio of the oil phase to the water phases, and the speed of homogenization. Higher concentrations of polymer and lower homogenization speeds yield larger particles. For a 1 micrometer particle, an oil/water ratio is typically 5 to 1. Sansdrap and Moes, J. Controlled Release 43: 47-58, 1997; Jeyanthi, et al., J. Microencapsulation 14: 163-174, 1997).

Poly-lactic acid (PLA) and poly-lactic-co-glycolic acid (PLGA) are two biodegradable polymers that are particularly suitable for making the microparticle. PLGA may be used for short-term delivery (days) and PLA may be used for long-term delivery (weeks or months), (Huh, et al., Drug Delivery Technology 2006, www.drugdeliverytech.com/cgi-bin/articles.cgi?idArticle=152).

The microparticle bears antibodies to endothelial cell surface proteins to allow the microparticle to “dock” on endothelial cells inside the blood vessels. A preferred endothelial cell surface protein for microparticle docking is endosialin, which is expressed in blood vessels surrounding a tumor, but not in the vicinity of normal tissue. (Rettig, et al., P.N.A.S. USA 89: 10832-10836, 1992; MacFadyen, et al., FEBS Letters 579: 2569-2575, 2005; Nanda, et al., P.N.A.S. USA 103: 3351-3356, 2006). Microparticles bearing surface antibodies to endosialin will selectively attach to endothelial cells in the vicinity of a tumor, thereby helping to target delivery of the Tag nanoparticles to tumor cells. However, any endothelial cell surface protein expressed in the vicinity of a tumor to which antibodies can be prepared may be used. The antibodies are covalently coupled to the microparticle by “functionalizing” the microparticle surface and the antibody with appropriate linkers using any appropriate method known in the art, e.g., methods recently reviewed by Torchilin, Advanced Drug Delivery Reviews 58: 1532-1555, 2006; and methods described by Yeo, et al., Chem. Eur. J. 10: 4664-4672, 2004; Fischer, et al., J. Controlled Release 111: 135-144, 2006; Bellocq, et al., Bioconjugate Chemistry 14: 1122-1132; Coombes, et al., Biomaterials 18: 1153-1161, 1997; and Blessing et al., Bioconjugate Chem. 12: 529-537, 2001.

### Therapeutic Nanoparticles

The therapeutic nanoparticles comprise therapeutic compounds incorporated into a biocompatible polymeric compound to prevent activation and/or degradation of the therapeutic compound before it reaches the target cell. Appropriate polymeric compounds may be prepared from any biocompatible, endosomolytic, cationic polymer including, but not limited to, polyethyleneimines, poly(amidoamine)s, histidine, polyhistidine, polylysine, poly(D,L-lactide-co-glycolide), polyglycolic acid, and combinations thereof. The therapeutic nanoparticle may range from about 100 nanometers to about 600 nanometers in diameter, preferably about 100 to about 200 nanometers in diameter.

Therapeutic compounds may include all classes of chemotherapeutic agents, i.e., alkylating agents, anti-metabolites, plant alkaloids and terpenoids, vinca alkaloids, podophyllotoxin, taxanes, topoisomerase inhibitors, apoptosis-inducing compounds, antibiotics, and hormones. Therapeutic compounds may also include compounds that stimulate cell proliferation and growth such as growth factors (e.g., epidermal growth factor, basic fibroblast growth factor), and angiogenic factors (e.g., vascular endothelial growth factor). The therapeutic compound may be in the form of a nucleotide that encodes a therapeutic agent, which is operably linked to a promoter that is active in eukaryotic cells, such as a plasmid vector bearing the sequence for a growth factor and an appropriate promoter sequence.

The therapeutic nanoparticles may be produced by methods known in the art, such as those described below, and in Boussif et al., P.N.A.S. USA 92: 7297-7301, 1995; Blessing et al., Bioconjugate Chem. 12: 529-537, 2001; Forrest, et al., Bioconjugate Chem. 14: 934-940, 2003; and Sullivan, et al., Gene Therapy 10: 1882-1890, 2003. Anti-Tag antibodies may be positioned on the surface of the therapeutic nanoparticles by covalent coupling as described, for example, in Blessing et al., Bioconjugate Chem. 12: 529-537, 2001; or by native chemical ligation, e.g., as described by Yeo, et al., Chem. Eur. J. 10: 4664-4672, 2004; or by any other appropriate means.

Treatment of Diseases with the Tag and Taraet Delivery System

As described above, the Tag and Target system may be administered intravenously to treat tumors. However, the system may also be used in conjunction with surgical methods to directly tag and treat tumor tissues or resected organs as described below under “Surgical treatment of tumor cells.” The Tag and Target system is particularly advantageous for tumor treatment, because it will not only destroy primary tumor cells, it will also tag and target metastatic cells, thereby reducing secondary tumor formation and cancer recurrence. In the event of recurrence, the tumor cells arising from the original tumor will already be tagged, enabling the targeted delivery of subsequent therapy. The Tag and Target system may also be used to inhibit tumor growth by inhibiting angiogenesis as described below under “Inhibition of angiogenesis.”

Coronary disease may be treated by using the system to stimulate angiogenesis as described under “Stimulation of angiogenesis.” The system may be used to stimulate regeneration in diseased organs such as liver, heart, and kidney, as described under “Stimulation of tissue regeneration,” by placing antibodies to organ-specific antigens on the Tag nanoparticle and targeting specific growth factors and other proliferation-stimulating compounds to these organs.

### Methods of Preparation

These methods describe preparation of possible embodiments of components of the Tag and Target system. Those of ordinary skill in the art will realize that these methods can be modified or other methods employed to prepare vectors, nanoparticles and microparticles suitable for the invention.

1. Preparation of Tag Vector

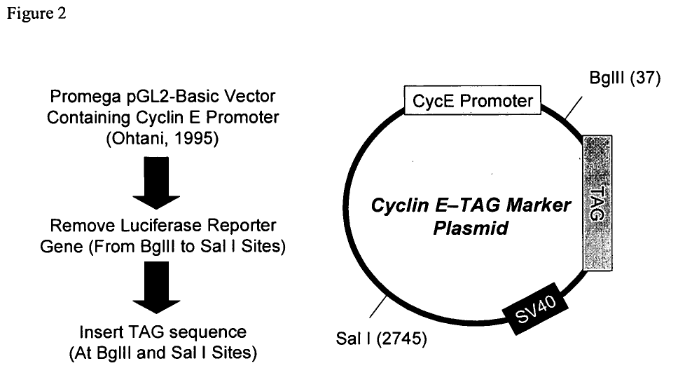

A generalized embodiment of a Tag vector is shown in FIG. 2. A nucleotide sequence corresponding to the human cyclin E promoter sequence (GenBank Accession No. L48996) is cloned into the HindIII (bp 37) and BgIII (bp 47) sites of the pGL2-Basic vector (Promega Corp.) as described by Ohtani, et al., P.N.A.S. USA 92: 12146-12150, 1995.

The luciferase expression nucleotide sequence is then removed from the pGL2-Basic plasmid containing the cyclin E promoter by restriction digest and the nucleotide sequence for the Tag peptide is ligated into the plasmid in place of the luciferase sequence. The plasmid containing the cyclin E promoter and the Tag sequence is then propagated in an appropriate strain of E. coli, and purified using methods well-known in the art.

The Tag nucleotide may comprise the combined nucleotide sequences of the FLAG® peptide and junctional adhesion molecule A (JAM-A, aka JAM-1), oriented to yield a fusion protein of JAM-A having the FLAG® peptide at the N-terminal. Fusion protein sequences may be prepared by methods well-known in the art. Commercially available plasmids with the FLAG® sequence, (FLAG® Protein Expression Systems, Sigma-Aldrich), may be used to prepare the fusion-protein sequence. A fusion construct of FLAG® or any other selected peptide sequence and JAM-A may also be created as a Tag. JAM-A is a cell-surface glycoprotein normally expressed on platelets. The JAM-A sequence ensures that the FLAG® or other Tag peptide will be expressed on the external surface of tagged cells. (Naik, et al., J. Cell Science 114: 539-547, 2000).

2. Preparation of Tag Nanoparticles

The cyclin E-JAM-A/FLAG vector described above is complexed with polyethylenimine following the method of Blessing, et al., Bioconjugate Chem. 12: 529-537, 2001), basically as follows. A 1.0 g/ml stock solution of anhydrous PEI (average MW of 25 kD) in 0.15M sodium chloride, pH 7.4 is prepared. To prepare a ratio of PEI amine to DNA phosphate (N:P) of 10, 10 ug of the DNA vector is diluted into 500 ul of 0.15M NaCl, and 500 ul of PEI stock is added drop-wise while vortexing at low speed. The condensation reaction is allowed to proceed at room temperature for 10 minutes for the formation of PEI-DNA complexes.

Bifunctional polyethylene glycol (PEG) (either α-vinyl sulfone-ω-N-hydroxy succinimide ester poly-(ethylene glycol), α-maleimide-ω-N-hydroxysuccinimide ester poly-(ethylene glycol), or α,ω-disuccinimidyl succinate terminated poly-(ethylene glycol)) (4 ug in HEPES buffered saline) is added to the PEI-DNA complexes. The mixture is allowed to stand at room temperature for approximately 30 minutes to allow the amine groups of PEI and succinimide groups of PEG to bind, forming PEI-DNA-PEGylated complexes. Epidermal growth factor is functionalized with a sulfhydryl group as described in Blessing, et al., to form EGF-SH. The EGF-SH (144 picomoles in 0.15M NaCl) is added to the PEGylated complexes and the mixture is left overnight at room temperature, allowing binding between the SH group at the end of the EGF molecule and the maleimide or succinimide group at the distal end of the PEG molecule. The resulting Tag nanoparticles bearing EGF on an outer surface may be stored at −20° C.

PEG attachment to the PEI-DNA complexes may be assessed through photon correlation spectroscopy. Conjugation of the EGF ligand to the polymer may be verified by determining the presence of particular amino acids via infrared spectroscopy. Nanoparticle morphology and ligand binding may also be characterized by atomic force microscopy. Nanoparticle size may be determined by any appropriate means, such as dynamic light scattering followed by atomic force microscopy for confirmation.

3. Preparation of Microparticles

Microparticles may be formulated by a modified multiple emulsion/solvent evaporation method (as described by De Rosa, et al., Journal of Pharmaceutical Sciences 91: 790-799, 2002)). Briefly, 400 ul of PEI/DNA complexes in NaCl or HEPES buffer at an N/P of 10 are emulsified into 2.5 ml of methylene chloride containing 250 mg of Poly(D,L-lactide-co-glycolide) (MW=98 kD), (“PLGA”). The solution is emulsified by homogenizing at 13,500 rpm for 2 minutes. The emulsion is then rapidly added into a solution containing 20 ml of 0.5% poly(vinylalcohol) and homogenized at 8,000 rpm for 1 minute. The resulting emulsion is stirred at 1000 rpm for 3 hours at room temperature to allow solvent evaporation. Microparticles are collected by centrifugation and then lyophilized for 48 hours at a pressure of less than about 1 mbar on a pre-cooled shelf at −40° C. Freeze-dried microparticles may be stored at 4° C.

Polyethylene glycol chains and targeting functionalities, such as antibodies to endosialin, may be added to the microparticle surface, as described above for nanoparticles, either before or after lyophilization. These functionalized microparticles are stored at −20° C.

Microparticles are examined for size distribution by dynamic light scattering and for morphology by scanning electron microscopy or atomic force microscopy, using standard methodology. The presence of DNA in the microparticles may be assessed by applying a DNA stain. The distribution of DNA complexes within the microparticles may also be assessed by labeling the DNA complexes with a fluorescent molecule before formation of the microparticle. The DNA complexes within the microparticles may then be viewed by fluorescence or confocal microscopy. Microparticles bearing fluorescent DNA particles may also be used to examine and optimize release and uptake of the DNA particles by cultured cells, as described in De Rosa, et al., Journal of Pharmaceutical Sciences 91: 790-799, 2002, and to analyze cell-surface expression of the Tag peptide.

4. Preparation of Therapeutic Nanoparticles

Therapeutic nanoparticles targeted for tumors may contain any of a number of chemo-therapeutic agents, for example, taxol, a microtubule destabilizing agent which is frequently used in chemotherapy. PLGA (100 mg) (MW=98 kD), paclitaxel (taxol) (10 mg), and isopropyl myristate (30 mg) are dissolved in 5 ml of methylene chloride. The organic phase is emulsified into a 50 ml 0.5% poly(vinylalcohol) solution by probe sonication in an ice bath, to form polymeric particles containing paclitaxel. The solution is further agitated by stirring at 800 rpm for 5 hours at room temperature to facilitate removal of the organic solvent. Polyethylene glycol chains and targeting functionalities are then added to the particle surface as described as described above in Methods of Preparation (2) for nanoparticles. TAGs comprising FLAG®-fusion proteins may be targeted with commercially available antibodies to the FLAG® peptide (Sigma-Aldrich, St. Louis, Mo.) Therapeutic nanoparticles may be stored at −20° C.

### Prospective Methods of Therapeutic Use

1. Parenteral Treatment of Tumor Cells

Following surgical removal of a tumor, the Tag and Target system may be administered intravenously in an appropriate vehicle, such as buffered saline. Tag nanoparticles are loaded into microparticles targeted for endosialin, as described in Method 3. The microparticles formulated via the parameters specified in Method 3 will achieve 80% release of the Tag nanoparticles in approximately 30-40 days (De Rosa, et al., Journal of Pharmaceutical Sciences 91: 790-799, 2002). Alternatively, the Tag nanoparticles may be directly administered intravenously.

The Tag nanoparticles are taken up by tumor cells and the Tag vector is incorporated into the cell genome during cell division. This completes Stages 1-3 of the Tag and Target treatment (FIG. 1). The concentration of microparticles to administer will depend on characteristics of the patient (e.g., age, weight, health) and the extent of disease. The release profile may be optimized by adjusting the molecular weight of the polymer and the amount of DNA incorporated.

In Stage 4 of the Tag and Target treatment (FIG. 1), therapeutic nanoparticles targeting the Tag peptide are administered intravenously in a physiologically compatible solution. After binding to the Tag peptides on the surfaces of tumor cells, the therapeutic nanoparticles are taken up by the tumor cells via endocytosis, and the therapeutic compound, e.g., taxol, is released intracellularly. The effects of Tag and Target treatment are assessed by monitoring changes in the course of the disease in the treated patient. Administration of therapeutic nanoparticles may be repeated as necessary.

2. Surgical Treatment of Tumor Cells

Following surgical removal of a tumor, surgeons may apply the Tag nanoparticles directly to tissues in the surgical site. Direct administration to these tissues increases the likelihood of labeling any remaining cancer cells with the Tag, and provides a way to subsequently inhibit metastasis and secondary tumor formation, thereby enhancing the therapeutic effects of the Tag and Target system.

Therapeutic nanoparticles targeted to the tagged cells may be subsequently administered to destroy remaining cancer cells. Should secondary tumors arise later, the tumor cells will already be tagged and therapeutic nanoparticles may again be administered parenterally to destroy them.

3. Inhibition of Angiogenesis

Tumor growth may be inhibited by blocking angiogenesis and “starving” the tumor cells. The Tag and Target delivery system may be used, generally as described in “Parenteral treatment of tumor cells,” for targeted delivery of compounds which inhibit angiogenesis, such as antibodies against vascular endothelial growth factor. For example, sites of angiogenesis may be recognized by antibodies or ligands that bind to endosialin, a peptide which is expressed by endothelial cells in blood vessels adjacent to tumor tissue.

4. Stimulation of Angiogenesis

Ischemic coronary artery diseases lead to heart attack and stroke when blood flow is severely constricted. As plaque accumulates in arteries, new blood vessel development is triggered due to ischemia, but proceeds very slowly. The Tag and Target delivery system may be used may be used, generally as described above in Prospective Methods of Therapeutic Use 1, for targeted delivery of angiogenic growth factors, such as vascular endothelial growth, to regions of new blood vessel growth to accelerate the formation of functional vessels around blocked arteries. New vessels allow blood flow to continue and can deter heart attack or stroke. Regions of new blood vessel development may be selected for tagging by labeling Tag nanoparticles with antibodies or ligands that bind to endothelial cell adhesion molecules such as p-selectin, ICAM and VCAM, which are highly expressed in endothelial cells in sites of ischemia.

5. Stimulation of Tissue Regeneration

Tissue and organ specific markers may be used to target tissues and organs for regeneration. The Tag and Target delivery system may be used, generally as described in “Parenteral treatment of tumor cells,” for targeted delivery of growth factors to stimulate and accelerate the formation of new tissue and blood vessels in the regeneration process. Growth factors that may be used to promote regeneration include, but are not limited to, acidic fibroblast growth factor, basic fibroblast growth factor, bone morphogenetic protein, epidermal growth factor, hepatocyte growth factor, hydroxyapatite, insulin-like growth factor I, nerve growth factor, platelet-derived growth factor, transforming growth factor, and vascular endothelial growth factor. The use of these growth factors in tissue regeneration has been widely studied. (Reviewed in Tabata, Tissue Engineering 9: Suppl. 1, S1-S15, 2003).

Regions of growth factor release may be selected for tagging by labeling nanoparticles with antibodies or ligands that bind to endothelial cell adhesion molecules, such as p-selectin, ICAM, and VCAM. These molecules are highly expressed in endothelial cells in ischemic sites, which are indicative of regions in need of tissue regeneration.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

### EXAMPLES

### 1. Development of Cell Cultures for Testing Cell-Cycle Dependent Expression of Tag Vector

Chinese hamster ovary (CHO) cells were obtained from the ATCC and maintained under standard culture conditions in DMEM medium containing 10% fetal bovine serum, 100 U/ml penicillin and 100 ug/ml streptomycin. To synchronize the cell cycles of the cultured cells, lovastatin was reconstituted in DMSO and added to the culture medium to a final concentration of 40 uM. In FIG. 3A cells were stained with propidium iodide to demonstrate the effects of treatments on cell cycle synchronization. The first panel shows the effects of DMSO alone and the middle panel shows that the number of cells in the G0/G1 stage of the cell cycle after 32 h of treatment with lovastatin is increased compared with DMSO treatment alone.

Addition of mevalonic acid lactone (to 5 mM) released the G0/G1 arrest, allowing the majority of cells to enter S phase synchronously within 24 h (FIG. 3A lower panel). This synchronized cell-cycle cell culture model was used to examine the timing of expression of a cell cycle-dependent promoter.

A cyclin E promoter-luciferase construct similar to that shown in FIG. 2, but with the luciferase sequence in place of a Tag sequence was transfected into the CHO cells via lipofectamine using standard transfection methods. An SV-40-Renilla expressing vector was used to normalize luciferase expression. FIG. 3B demonstrates that, under the effect of lovastatin, expression of the cyclin E-luciferase construct was inhibited; but when the cells were released from the lovastatin-induced G0/G1 block with mevalonic acid lactone, expression of the cyclin E-luciferase construct was stimulated. Similar results were obtained using a vector containing the cyclin E promoter and green fluorescent protein (GFP), as shown in FIG. 3C (blue—autofluorescence in non-transformed cells; green—lovastatin blocked cells; red—lovastatin treated cells 24 h after treatment with mevalonic acid lactone). These experiments demonstrate the feasibility of utilizing promoters that are specifically expressed only in cells that are committed to divide to tag dividing cells, such as tumor cells.

### 2. Preparation of PEI-DNA-PEG Nanoparticles

PEI-DNA-PEG nanoparticles were prepared according to the method of Blessing, et al., Bioconjugate Chem. 12: 529-537, 2001), basically as follows. A DNA solution of 500 ul was formulated with 20 ug of a PNA-binding vector. PNA is a “peptide nucleic acid” analog, commercially available from Panagene in South Korea, that is capable of binding DNA. The PNA-binding vector can bind fluorescently-labeled PNA (PNA-F) to allow intracellular detection of the vector. A PEI (poly(ethylenimine)) solution of 500 ul was formulated with 60 ul of 0.45 mg/ml 25 kD PEI. The PEI solution was added to the DNA solution in a drop-wise manner while vortexing at low to medium speed. The condensation reaction is allowed to proceed at room temperature for 10 minutes to allow the formation of PEI-DNA complexes.

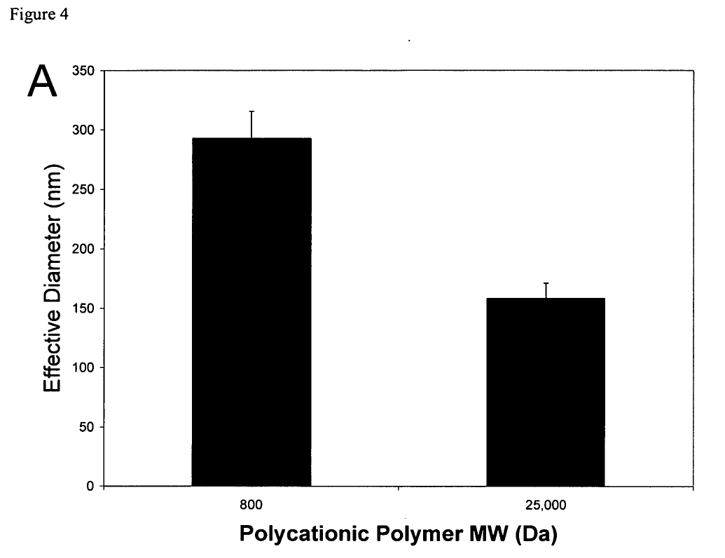

Dynamic light scattering and atomic force microscopy were used to characterize the PEI-DNA complexes as shown in FIGS. 4 A and B. In this experiment, the PEI-DNA complexes ranged in size from 120 to 180 nm in diameter.

Bifunctional polyethylene glycol (PEG) (α,ω-disuccinimidyl succinate terminated poly-(ethylene glycol), 0.4 mg/mL in 20 mM HEPES buffer) was added to the PEI-DNA complexes to increase colloidal stability. A PEG:DNA weight ratio of 20 was found to be optimal. The mixture was allowed to stand at room temperature for approximately 30 minutes to allow the amine groups of PEI and succinimide groups of PEG to bind, forming PEI-DNA-PEGylated complexes.

### 3. Transfection of Cells with PEI-DNA Nanoparticles

CHO cells were transfected with the PEI-DNA nanoparticles prepared according to Example 2. DNA consisted of the PNA-F-bound gWIZ-GFP (Genlantis, San Diego, Calif.) vector with no added exogenous promoter. FIG. 4C shows PNA-F fluorescent signals within CHO cells transfected with fluorescently-labelled PEI-DNA-PEG complexes, demonstrating that PEI-DNA-PEG nanoparticles can be used to deliver exogenous gene sequences to cells.

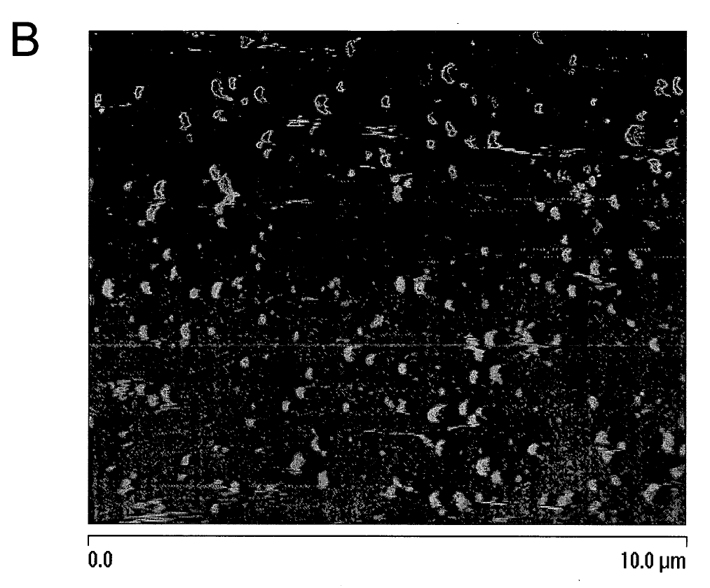

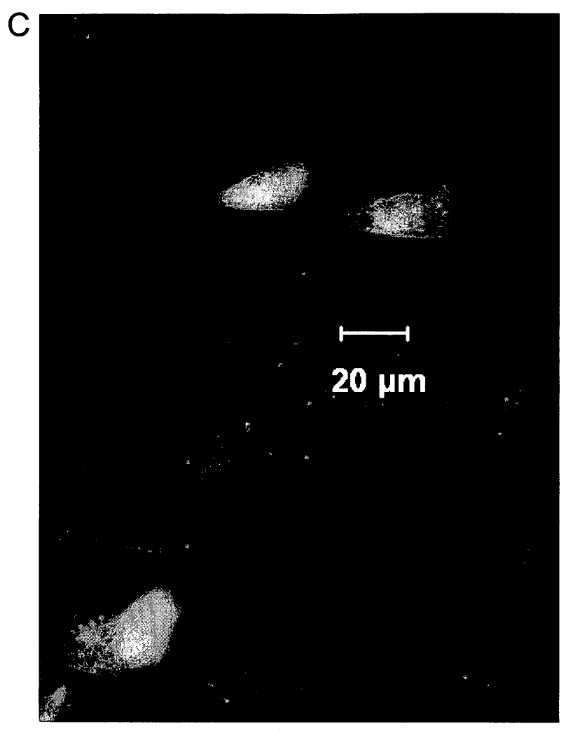

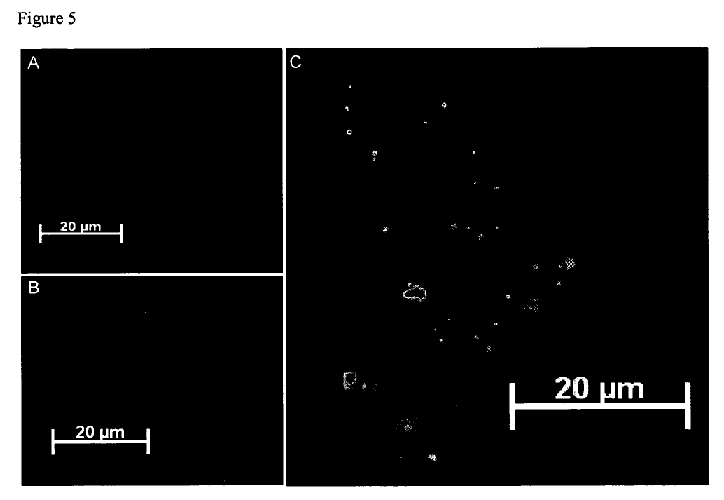

FIG. 5 also demonstrates localization of the nanoparticles within the perinuclear region of the cells three hours after transfection. FIG. 5A shows cells transfected with unlabelled PEI-DNA nanoparticles to assess autofluorescence. FIG. 5B shows cells transfected with fluorescently-labelled PNA (PNA-F) alone, and demonstrates that free PNA-F does not enter the cells. FIG. 5C shows cells transfected with PEI-DNA (gWIZ-GFP) bound to PNA-F and demonstrates the effectiveness of these nanoparticles in transfecting cells. Blue—Hoechst 33258 nuclear stain, green—PEI-DNA-PNA-F nanoparticles.

Transfection efficiency could be enhanced by coating the PEI-DNA-PEG nanoparticles with the integrin-binding peptide, RGD (arginine, glycine, aspartic acid). The surface of PEI-DNA-PEG nanoparticles was saturated with the peptide of sequence YYYGRGDSP via binding of the N-terminus of the peptide to the PEG chain through succinimide chemistry. Briefly, PEI-DNA-PEG nanoparticles prepared as described in Example 2, were incubated at room temperature for 30 min with a 1:1 molar ratio of YYYGRGDSP peptide.

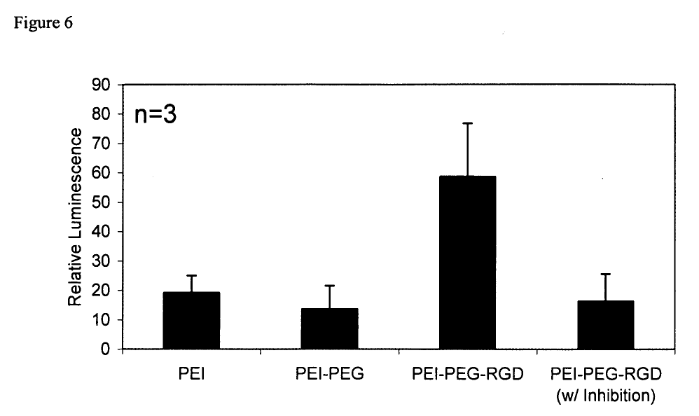

FIG. 6 demonstrates that the addition of RGD peptide increased transfection efficiency approximately four-fold compared with PEI-PEG alone. In addition, when an excess of free RGD peptide was added to the cell culture medium prior to transfection to block subsequent RGD binding to cells, transfection efficiency was inhibited as shown in FIG. 6. These results demonstrate that specifically-binding molecules, such as integrin-binding peptides, could be used to target PEI-DNA-PEG nanoparticles to tumor tissue.

### 4. Uptake of PEI-DNA-PEG-RGD Nanoparticles by Tumor Cells In Vivo

PEI-DNA-PEG-RGD nanoparticles were prepared by combining 50 ug DNA and 150 ul of 0.45 mg/ml 25 kD PEI as described above. The particles were then PEGylated and saturated with RGD peptide as described above. Concentration of “nanoparticles” is measured as amount of DNA. Nanoparticles created with 50 ug DNA (cyclin E promoter-GFP construct) were concentrated by filtration from 2.5 ml total volume to a final volume of 100 ul for in vivo administration.

Tumors were initiated in 5 month old C57BL/6 mice by dorsal subcutaneous injection of 1×10⁶ B16FO mouse melanoma cells per mouse. Mice were assessed visually for tumor formation after two weeks, at which time the average tumor diameter was approximately 11 mm.

One group of three mice were injected via tail vein with 50 ug of the DNA vector construct containing the cyclin E promoter and GFP sequences (“naked” DNA). A second group of three mice were administered 100 ul of nanoparticles (PEI-DNA-PEG-RGD) prepared with 50 ug of the same DNA vector construct by injection directly into the tumor. A control group of three mice was treated by injection of 20 mM HEPES buffer into the tail vein to measure autofluorescence. Animals were sacrificed after 48 hours. Tumors were removed and the tumor cells disaggregated by incubation with bacterial collagenase at 37° C. for 1 h. Expression of GFP by living cells was analyzed by flow cytometry using a FACSCalibur™ (BD Biosciences) fluorescent intensity analyzer.

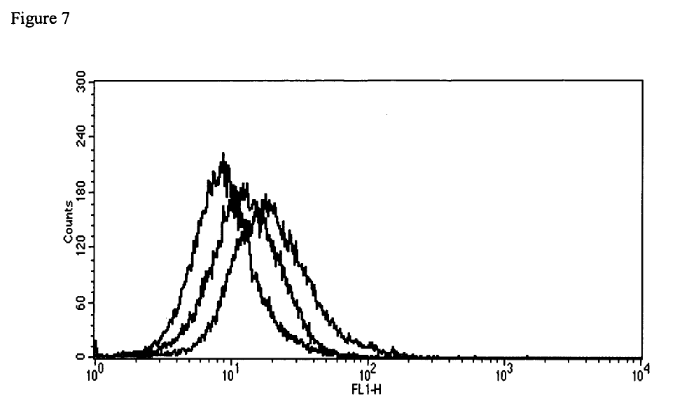

FIG. 7 demonstrates that both “naked” DNA constructs delivered through the tail vein (red) and PEI-DNA-PEG-RGD nanoparticles delivered directly into the tumor (green) expressed GFP, and these peaks were shifted relative to the peak of background autofluorescence in control mice (blue).

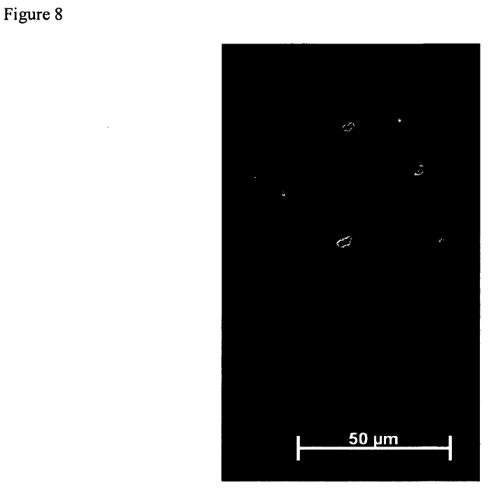

FIG. 8 shows expression of GFP within disaggregated tumor cells from tumors injected directly with PEI-DNA-PEG-RGD nanoparticles. Disaggregated cells were plated on gelatin-coated substrates and analyzed by fluorescence microscopy. These experiments demonstrate that PEI-DNA nanoparticles can be used in vivo to specifically tag tumor cells for subsequent targeted delivery of therapeutic nanoparticles.

SEQUENCE LISTING

| <110> Naik, Ulhas | |
|---|---|
| Mellili, Peter | |
| <120> Tag and Target Delivery System | |
| <130> UOD 140USP | |
| <160> 1 | |
| <170> PatentIn version 3.3 | |
| <210> 1 | |
| <211> 292 | |
| <212> DNA | |
| <213> Homo sapiens | |
| <400> 1 | |
| caggccctgt cacttggccc cgccctgtcc gccggccccg cccctgattc cccgtccctg | 60 |
| cgcctcgctg gccggcgccg tggaggggcg ggtccggggg cggggcgagg gacggggcgg | 120 |
| gacgggctct gggtcccgcg cggccgctga ggggctggga gccgcggcgg ggcggtgcga | 180 |
| gggcgggccg gggccggttc cgcgcgcagg gattttaaat gtcccgctct gagccgggcg | 240 |
| caggagcagc cggcgcggcc gccagcgcgg tgtagggggc aggcgcggat cc | 292 |

What is claimed:

1. A composition for selectively tagging a dividing cell for subsequent targeted delivery of a therapeutic compound to a subject in need thereof, comprising a plurality of Tag nanoparticles, each Tag nanoparticle comprising a nucleotide operably linked to a promoter that functions only in dividing cells, wherein the nucleotide encodes a Tag peptide.

2. The composition of claim 1, wherein the Tag peptide is selected from the group consisting of a xenogenic peptide, a peptide expressed only by a target cell, and a nonsense peptide.

3. The composition of claim 1, wherein each of the Tag nanoparticles further comprises a polymer.

4. The composition of claim 1, wherein the Tag nanoparticles further comprise a plurality of antibodies on an external surface of the nanoparticle.

5. The composition of claim 1 further comprising a microparticle, wherein the microparticle contains the plurality of Tag nanoparticles.

6. The composition of claim 5, wherein the microparticle further comprises a plurality of antibodies on an external surface of the microparticle.

7. The composition of claim 4, wherein the antibodies are specific for an extracellular domain of at least one peptide or protein expressed by a tumor cell or by an endothelial cell in the vicinity of a tumor cell.

8. The composition of claim 6, wherein the antibodies are specific for an extracellular domain of at least one peptide or protein expressed by endothelial cells.

9. The composition of claim 1, wherein the promoter that functions only in dividing cells comprises the nucleotide sequence of SEQ ID NO:1.

10. The composition of claim 1, wherein the promoter that functions only in dividing cells is selected from the group consisting of promoters for the A cyclins, the B cyclins, the C cyclins, the D cyclins, the E cyclins, the F cyclins, the G cyclins, and E2F Transcription Factor.

11. A method of selectively tagging a dividing cell, comprising administering the composition of claim 1 to the dividing cell.

12. The method of claim 11, wherein the dividing cell is selected from the group consisting of a cancer cell, an endothelial cell, a liver cell, a heart cell, and a kidney cell.

13. A method for targeted delivery of a therapeutic compound to a subject in need thereof, comprising the steps of (1) tagging a dividing cell in the subject by administering to the subject a composition comprising one or more Tag nanoparticles, each of the Tag nanoparticles comprising a nucleotide operably linked to a promoter that functions only in dividing cells, wherein the nucleotide encodes a Tag peptide; and (2) subsequently administering to the subject a therapeutic nanoparticle comprising an effective amount of a therapeutic compound, wherein the therapeutic nanoparticle has an external surface comprising one or more ligands capable of specifically binding to the Tag peptide.

14. The method of claim 13, wherein the therapeutic nanoparticle further comprises a polymer.

15. The method of claim 13, wherein the therapeutic compound is selected from the group consisting of alkylating agents, anti-metabolites, plant alkaloids, plant terpenoids, vinca alkaloids, podophyllotoxin, taxanes, topoisomerase inhibitors, apoptosis-inducing compounds, anti-proliferative compounds, immune activators, antibiotics, and hormones.

16. The method of claim 13, wherein the therapeutic compound is selected from the group consisting of growth factors and angiogenic factors.

17. The method of claim 13, wherein the therapeutic compound is administered as a nucleotide that is operably linked to a eukaryotic promoter, wherein the nucleotide encodes a therapeutic compound.

18. A method of treating a cancer cell and inhibiting metastasis in a subject in need thereof, comprising the steps of (1) tagging a cancer cell by administering the composition of claim 1 to the subject; (2) delivering to the subject a therapeutic nanoparticle capable of recognizing the tagged cell, and (3) subsequently repeating step (2) or steps (1) and (2).

19. The composition of claim 5, wherein the microparticle has a diameter of from about 600 nanometers to about 10 micrometers.

20. The composition of claim 19, wherein the microparticle has a diameter of from about 1 to about 5 micrometers.

21. The composition of claim 1, wherein the Tag nanoparticle has a diameter of about 600 nanometers or less.

22. The composition of claim 21, wherein the Tag nanoparticle has a diameter of from about 50 to about 400 nanometers.

23. The method of claim 13, wherein the therapeutic nanoparticle has a diameter of 600 nanometers or less.

24. The method of claim 11, wherein the composition is administered intravenously.

25. The method of claim 11, wherein the composition is administered directly to tissue in a surgical site.

26. The method of claim 13, wherein the therapeutic nanoparticle is administered intravenously.

27. The method of claim 13, wherein the therapeutic nanoparticle is administered directly to tissue in a surgical site.

28. A method of selectively tagging a dividing cell in a surgical site for subsequent targeted delivery of a therapeutic compound to a subject in need thereof, comprising administering the composition of claim 1 to the surgical site.

29. The composition of claim 1, wherein the Tag peptide comprises a peptide or protein selected from the group consisting of FLAG® peptide, interleukin-2, and JAM-A.
